(19) United States
(12) Patent Application Publication
(10) Pub. No.: US 2023/0010556 A1
(43) Pub. Date: Jan. 12, 2023

(54) TRANSDERMAL COLD ATMOSPHERIC PLASMA-MEDIATED IMMUNE CHECKPOINT BLOCKADE THERAPY

(71) Applicant: THE REGENTS OF THE UNIVERSITY OF CALIFORNIA, Oakland, CA (US)

(72) Inventors: Zhen Gu, Los Angeles, CA (US); Richard E. Wirz, Los Angeles, CA (US); Guojun Chen, Los Angeles, CA (US); Zhitong Chen, Los Angeles, CA (US)

(73) Assignee: THE REGENTS OF THE UNIVERSITY OF CALIFORNIA, Oakland, CA (US)

(21) Appl. No.: 17/779,903

(22) PCT Filed: Dec. 10, 2020
(86) PCT No.: PCT/US2020/064361

§ 371 Date: May 25, 2022

Related U.S. Application Data
(60) Provisional application No. 62/947,303, filed on Dec. 12, 2019.

(51) Int. Cl. A61M 37/00; A61N 1/44; A61K 39/395; A61K 9/00; A61P 35/00

(52) U.S. Cl. CPC A61M 37/0015; A61N 1/44; A61K 39/3955; A61K 9/0021; A61P 35/00; A61K 9/0092; A61M 2037/0007; A61M 2037/0023; A61M 2037/003; A61M 2037/0061

(57) ABSTRACT

A cold atmospheric plasma (CAP)-mediated ICB therapy/delivery device are disclosed herein that employs a patch having microneedles that are used to deliver the CAP transdermally along with an immune checkpoint inhibitor for enhancing transdermal treatment efficacy. The hollow-structured microneedle patch can facilitate the transportation of CAP through the skin, causing tumor cell death. The release of cancer antigens then promotes the maturation of dendritic cells in the tumor-draining lymph nodes, subsequently initiating the T cell-mediated immune response. Anti-PDL1 antibody (aPDL1), an immune checkpoint inhibitor (or other immune checkpoint inhibitors), released from the microneedle patch (in some embodiments) further augments the anti-tumor immunity. The transdermal combinational CAP and ICB therapy inhibits tumor growth for both primary tumors as well as distant tumors, with prolonged survival in the tumor-bearing mice. Such results should translate to other species.

### RELATED APPLICATION

This application claims priority to U.S. Provisional Patent Application No. 62/947,303 filed on Dec. 12, 2019, which is hereby incorporated by reference in its entirety. Priority is claimed pursuant to 35 U.S.C. § 119 and any other applicable statute.

### STATEMENT REGARDING FEDERALLY SPONSORED RESEARCH AND DEVELOPMENT

This invention was made with government support under Grant Number CA234343 awarded by the National Institutes of Health. The government has certain rights in the invention.

### TECHNICAL FIELD

The technical field generally relates to therapeutic uses of cold atmospheric plasma (CAP) against cancer in combination with immune checkpoint blockade (ICB) therapy. More particularly, the technical field relates to a microneedle-containing patch that is used to deliver or transport CAP through skin causing cancer cell death. The therapy is optionally combined with an immune checkpoint inhibitor which is released from the patch to further augment the anti-tumor immunity.

### BACKGROUND

The immune checkpoint blockade (ICB) is known to increase antitumor immunity by inhibiting intrinsic down-regulators of immunity, which greatly transforms human cancer therapeutics. Various immune checkpoint inhibitors (ICI) have been identified and used in immunotherapy applications against cancer. Despite the exciting clinical outcomes, the overall objective rate of ICB remains to be improved. Meanwhile, the existing severe side effects associated with ICB also emphasize the essential need for new delivery approaches for ICB therapeutics. Local delivery of ICI to the targeted sites could be a desirable approach to minimize those limitations and augment the therapeutic efficacy.

Plasma, the fourth state of matter (solid, liquid, gas, and plasma), comprising over 99% of the visible universe, is an ionized gas composed of positive/negative charges, neutral atoms, radicals, and ultraviolet photons. The production of cold atmospheric plasma (CAP) under atmospheric pressure and room temperature has been used in a variety of applications including cancer therapy. The anti-cancer effect of CAP mainly relies on the synergistic action of the reactive oxygen species (ROS) and reactive nitrogen species (RNS). Currently, CAP, although promising, has unsatisfactory efficacy since the penetration of CAP towards tumor tissues is highly limited, which could also explain the necessity of multiple/frequent CAP treatments in order to achieve observable outcomes. Thus, both ICB and CAP have disadvantages for the treatment of mammalian cancers.

### SUMMARY

In one embodiment, a device or therapeutic system includes a patch having an array of microneedles (sometimes referred to as MN or MNs) formed thereon. The microneedles are hollow structures that permit the passage of CAP transdermally through tissue. The patch also includes, in some embodiments, one or more immune checkpoint inhibitors that are loaded in the microneedles and are released into the tissue to synergistically enhance treatment efficacy. The hollow-structured microneedle (hMN) patch has a void or hollow region that, together with the porous nature of the microneedle itself, forms effective “microchannels” within the microneedles to transport CAP through the skin or other tissue into tumors. Cancer cell death induced by CAP releases antigen and promotes dendritic cell (DC) maturation in the tumor-draining lymph node, where DCs can present the major histocompatibility complex-peptide to the T-cell receptor. The subsequent T-cell mediated immune response is initiated and can be further augmented by an immune checkpoint inhibitor such as anti-PDL1 antibody (aPDL1) from hMN patches. Thus, the synergism between CAP and ICB provides a broad platform technique for enhanced cancer treatment for both primary tumors and distant tumors.

In another embodiment, a method of treating cancer or other neoplasms in mammals includes applying a patch having a plurality of hollow microneedles formed therein or thereon to the tissue of the mammalian subject (e.g., skin tissue). CAP is then delivered to the tissue via the hollow microneedles. For example, using a patch that is applied to skin tissue, the CAP is delivered transdermally through the skin tissue. In addition, portions of the patch and/or hollow microneedles can be loaded with one or more immune checkpoint inhibitors that synergistically interact with the CAP to augment anti-tumor immunity to improve the efficacy of cancer or other neoplasm treatment. In one embodiment, the one or more immune checkpoint inhibitors include anti-PDL1 antibody (aPDL1). Examples of immune checkpoint inhibitors that may be used in conjunction with the patch include, by way of example, ipilimumab, nivolumab, pembrolizumab, avelumab, atezolimumab, durvalumab.

In one embodiment, the patch device is operably coupled to or interfaced with a CAP delivery device that includes a nozzle or dispensing head that includes two (or more) electrodes connected to a high voltage power source which is used to generate the CAP. A feed gas or vapor, which may include helium, is fed through the gas delivery device and is exposed to the two (or more) electrodes to generate the CAP that then exits the nozzle or dispensing head and passes through the hollow microneedles to reach the tissue containing the patch. The patch may be made from a number of materials including biocompatible polymers that may, for example, include a mixture of two biocompatible polymers, polyvinylpyrrolidone (PVP) and polyvinyl alcohol (PVA). In this particular example, PVP supports the strong mechanical strength of microneedles and PVA slows down the dissociation of microneedle patches upon fluids, though other materials may be used to perform these functions.

In some embodiments, the patch is used to treat diseased or cancerous tissue directly. That is to say the patch is applied to place the microneedles directly in contact with or immediately adjacent to the diseased tissue. In other embodiments, however, distant diseased tissue (e.g., distant tumors) may also be treated with the patch applied to another location.

In another embodiment, a patch for therapeutic treatment of tissue includes a base having a plurality of hollow microneedles extending away from the surface of the base, wherein the plurality of hollow microneedles each have a hollow space or void extending partially through the respective microneedles. In some embodiments, the plurality of hollow microneedles further comprise one or more immune checkpoint inhibitors contained therein.

In another embodiment, a system for treatment of cancer or other neoplasms in mammals includes a patch comprising a plurality of hollow microneedles, wherein the plurality of hollow microneedles each have a hollow space or void extending partially through the respective microneedles, wherein the plurality of hollow microneedles further comprise one or more immune checkpoint inhibitors contained therein. The system further includes a cold atmospheric plasma (CAP) delivery device comprising a gas nozzle or dispensing head having two or more electrodes operatively coupled to a high voltage source.

Additional embodiments may include conditions or states where the patches may selectively be exposed to CAP for full durations, short durations, or not at all, where the treatment approach depends on the condition and response of the subject.

### BRIEF DESCRIPTION OF THE DRAWINGS

FIG. 1A schematically illustrates a system for transdermal cold atmospheric plasma-mediated immune checkpoint blockade therapy. The schematic illustrates the transdermal combinational CAP and ICB therapy assisted by the polymeric hollow-structured microneedle patch loaded with aPDL1. Th cell, T helper cell; Tc cell, cytotoxic T cell; TCR, T-cell receptor; MHC, major histocompatibility complex.

FIG. 1B illustrates top and side views of the hollow-structured microneedle patch according to one embodiment.

FIG. 2 illustrates one particular embodiment of a system in which a hand-held delivery device that is used to apply CAP to a hollow-structured microneedle patch secured to tissue (e.g., skin).

FIG. 3 illustrates the CAP experimental set-up configuration, including DC power supply, AC signal generator, high voltage probe (used only for experimental results), oscilloscope (used only for experimental results), transformer, a CAP jet assembly (with nozzle), and a gas feed tube.

FIGS. 4A-4D illustrate characterization of the CAP setup. (FIG. 4A) Typical discharge voltage, frequency, and waveform for the CAP delivery device. (FIG. 4B) Photograph of the generated CAP jet. (FIG. 4C) A typical OES spectrum of CAP. (FIG. 4D) Temperature measurement of the generated CAP.

FIG. 5A illustrates ROS concentration in the media after CAP treatment (n=3). Data are presented as mean±SEM.

FIG. 5B illustrates ROS concentration in the cells after CAP treatment (n=3). Data are presented as mean±SEM.

FIG. 5C illustrates RNS concentration in the media after CAP treatment (n=3). Data are presented as mean±SEM.

FIG. 5D illustrates RNS concentration in the cells after CAP treatment (n=3). Data are presented as mean±SEM.

FIG. 6A illustrates cell viability of Bl6F10 melanoma cells after CAP treatment for different time (n=4).

FIG. 6B illustrates in vitro activation of DCs (CD86⁺CD80⁺ cells) in response to CAP treatment for different time (n=3).

FIG. 6C illustrates representative flow cytometric analysis images of DC maturation induced by CAP treatment for different times. Data are presented as mean±SEM.

FIG. 7A illustrates representative SEM images and of hMN patch from views of needle side (left) and base side (right). Scale bar: 200 μm.

FIG. 7B illustrates a photograph showing the penetration test of CAP through hollow-structured microneedle (hMN) patch.

FIG. 7C illustrates representative OES spectra of CAP before the hMN patch.

FIG. 7D illustrates representative OES spectra of CAP after the hMN patch.

FIG. 8 illustrates a graph showing the mechanical property of the hMN patch. The mean failure force is 0.23 N/needle.

FIG. 9 schematically illustrates a method for fabricating the microneedle patch according to one embodiment.

FIG. 10A illustrates the OES spectra of original CAP.

FIG. 10B illustrates the OES spectra of CAP penetrating solid microneedles (sMN).

FIG. 10C illustrates the OES spectra of CAP penetrating the mouse skin.

FIG. 10D illustrates the OES spectra of CAP penetrating the mouse skin applied with hMN.

FIG. 11 illustrates electron micrograph images of hMN patches after CAP treatment. No significant changes were observed. Scale bar: 200 μm.

FIG. 12 illustrates a graph of in vitro aPDL1 release profile from the hMN patch. Data are presented as mean±SEM. (n=3).

FIG. 13 illustrates a graph showing cell viability (%) as a function of microneedle (MN) concentration of the blank MNs. hMN patches were re-dissolved and added to Bl6F10 cells for 24 h of incubation. Data are presented as mean±SEM. (n=4).

FIG. 14 illustrates a graph of measurements of local temperature in the CAP-treated tumor area during CAP treatment in mice. Data are presented as mean±SEM. (n=4).

FIG. 15 illustrates a graph of the quantification of in vivo DC maturation in the tumor-draining lymph nodes of treated mice. Cells in the tumor-draining lymph nodes were collected three days after various treatments (G1: untreated; G2: CAP; G3: sMN/CAP; and G4: hMN/CAP) for assessment by flow cytometry after staining with CD11c, CD80 and CD86. Data are presented as mean±SEM (n=4). Statistical significance was calculated via one-way ANOVA with a Tukey post-hoc test. *P<0.05; **P<0.01; ***P<0.001.

FIG. 16 illustrates representative flow cytometric analysis of cells obtained in the tumor-draining lymph nodes collected as noted above with respect to FIG. 15.

FIG. 17 schematically illustrates the treatment schedule used for the mice.

FIG. 18 illustrates in vivo bioluminescence images of the untreated mice and mice treated with CAP, sMN/CAP, hMN/CAP, hMN-aPDL1, and hMN-aPDL1/CAP (aPDL1: 200 μg; CAP treatment: 4 min). Four representative mice per treatment group are shown.

FIG. 19 illustrates individual tumor growth kinetics in different groups (n=7). Growth curves were stopped when the first mouse of the corresponding group died. Data are presented as mean±SEM.

FIG. 20 shows average tumor growth kinetics in different groups (n=7) for the groups illustrated in FIG. 19. Data are presented as mean±SEM. Statistical significance was calculated via one-way ANOVA with a Tukey post-hoc test. *P<0.05; **P<0.01; ***P<0.001.

FIG. 21 illustrates Kaplan-Meier survival curves for treated and control mice (n=7). Statistical significance was calculated via the log-rank (Mantel-Cox) test. *P<0.05; **P<0.01; ***P<0.001.

FIG. 22 illustrates the intratumoral percentage of the CD3⁺ T cells in the Bl6F10 tumor three days after different treatments (n=4). Data are presented as mean±SEM. Statistical significance was calculated via one-way ANOVA with a Tukey post-hoc test for multiple comparisons. *P<0.05; **P<0.01; ***P<0.001.

FIG. 23 illustrates intratumoral percentage of the CD8⁺ T cells in the Bl6F10 tumor three days after different treatments (n=4). Data are presented as mean±SEM. Statistical significance was calculated via one-way ANOVA with a Tukey post-hoc test. *P<0.05; **P<0.01; ***P<0.001.

FIG. 24 illustrates intratumoral percentage of the CD4⁺ T cells in the Bl6F10 tumor three days after different treatments (n=4). Data are presented as mean±SEM. Statistical significance was calculated via one-way ANOVA with a Tukey post-hoc test. *P<0.05; **P<0.01; ***P<0.001.

FIG. 25 illustrates representative flow cytometric analysis of T cell infiltration gating on CD3⁺ cells within the tumors of CD4⁺ and CD8⁺ T cells in the tumor in different groups (primary tumor model). G1, untreated; G2, CAP; G3, sMN/CAP; G4, hMN/CAP; G5, hMN-aPDL1; G6, hMN-aPDL1/CAP.

FIG. 26 illustrates quantitative and representative flow cytometric analysis of Tregs cell within the tumors in different groups (primary tumor model). Data are presented as mean±SEM. (n=3). G1, untreated; G2, CAP; G3, sMN/CAP; G4, hMN/CAP; G5, hMN-aPDL1; G6, hMN-aPDL1/CAP. Statistical significance was calculated via one-way ANOVA with a Tukey post-hoc test. ***P<0.001.

FIG. 27 illustrates cytokine levels in the serum from mice isolated three days after different treatments (primary tumor model). Data are presented as mean±SEM. (n=5). G1, untreated; G2, CAP; G3, sMN/CAP; G4, hMN/CAP; G5, hMN-aPDL1; G6, hMN-aPDL1/CAP. Statistical significance was calculated via one-way ANOVA with a Tukey post-hoc test. *P<0.05; **P<0.01; ***P<0.001.

FIG. 28 illustrates a schematic illustration of the treatment schedule for CAP and hMN-aPDL1 for inhibiting distant tumor growth. Tumors on the right side were designated as “primary tumor” with combinational treatment, and tumors on the left side were designated as “metastatic tumor” without any treatment (n=7).

FIG. 29 illustrates in vivo bioluminescence images of the untreated mice and treated mice treated. Three representative mice per treatment group are shown.

FIG. 30 illustrates left and right tumor growth curves in untreated and treated mice. Data are presented as mean±SEM (n=7). Statistical significance was calculated via one-way ANOVA with a Tukey post-hoc test. *P<0.05; **P<0.01; ***P<0.001.

FIG. 31 illustrates left and right tumor weights in untreated and treated mice. Data are presented as mean±SEM (n=7). Statistical significance was calculated via one-way ANOVA with a Tukey post-hoc test. *P<0.05; **P<0.01; ***P<0.001.

FIG. 32 illustrates intratumoral percentage of the CD3⁺ T cells in the Bl6F10 tumor after different treatments (n=5). Data are presented as mean±SEM. Statistical significance was calculated via one-way ANOVA with a Tukey post-hoc test for multiple comparisons. *P<0.05; **P<0.01; ***P<0.001.

FIG. 33 illustrates intratumoral percentage of the CD8⁺ T cells in the Bl6F10 tumor after different treatments (n=5). Data are presented as mean±SEM. Statistical significance was calculated via one-way ANOVA with a Tukey post-hoc test. *P<0.05; **P<0.01; ***P<0.001.

FIG. 34 illustrates intratumoral percentage of the CD4⁺ T cells in the Bl6F10 tumor after different treatments (n=5). Data are presented as mean±SEM. Statistical significance was calculated via one-way ANOVA with a Tukey post-hoc test. *P<0.05; **P<0.01; ***P<0.001.

FIG. 35 illustrates quantitative and representative flow cytometric analysis of Tregs cell within the tumors in different groups (distant tumor model). Data are presented as mean±SEM. (n=4). Statistical significance was calculated via one-way ANOVA with a Tukey post-hoc test. **P<0.01; ***P<0.001.

FIG. 36 illustrates cytokine levels in the serum isolated three days after different treatments (distant tumor model). Data are presented as mean±SEM. (n=5). Statistical significance was calculated via Student's t-test. *P<0.05; **P<0.01.

### DETAILED DESCRIPTION OF THE ILLUSTRATED EMBODIMENTS

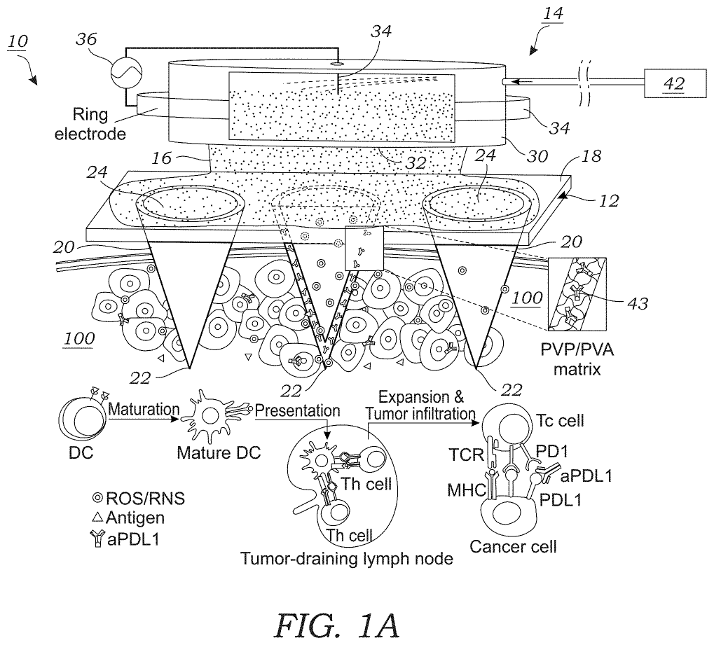

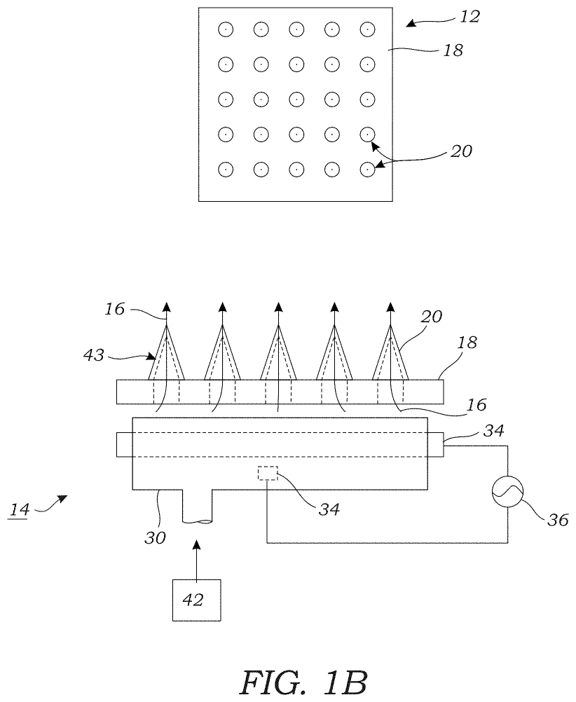

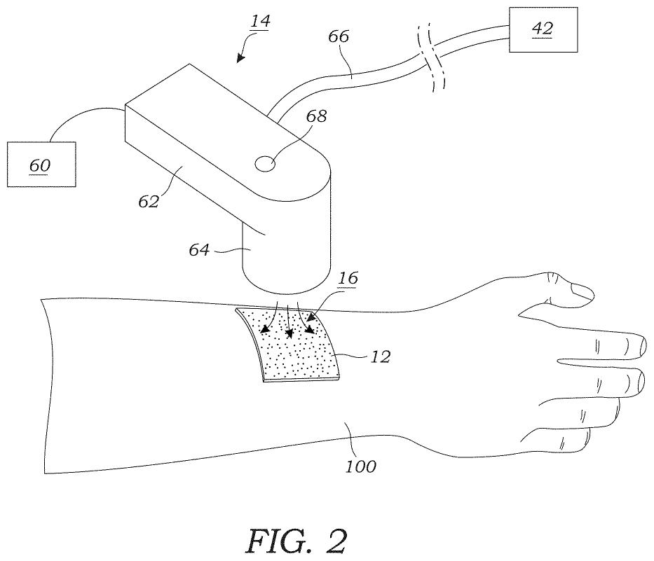

FIG. 1A is an illustration of a system 10 for transdermal cold atmospheric plasma-mediated immune checkpoint blockade therapy. The system 10 includes both a patch 12 and delivery device 14 for the delivery of cold atmospheric plasma (CAP) 16 to the applied tissue 100 via the patch 12. The tissue 100 may a number of types of tissue including, but not limited to, skin tissue. The tissue 100 may be diseased tissue and/or healthy tissue. FIG. 2 illustrates one particular embodiment of the system 10 in which a hand-held delivery device 14 is used to apply CAP 16 to patch 12 secured to tissue 100 (e.g., skin tissue as illustrated).

With reference to FIGS. 1A and 1B, the patch 12 includes a base 18 and a plurality of hollow microneedles 20 that extend outwardly from the base 18 to a tip 22. The hollow microneedles 20 may be located in an array such as that illustrated in FIG. 1B. The microneedles 20 are needle-like and include a hollow space or void 24 that is used for the passage of CAP 16 through the microneedles 20. Note that the hollow space or void 24 (FIGS. 1A and 1B) formed in the microneedles 20 does not fully extend to the tip 22 of the microneedles 20. Instead, the hollow space or void 24 extends from the back of the patch 12 (or back of the microneedles 20) and extends partially into the microneedle volume but does not have an exit (like a nozzle of a hose). The hollow space or void 24 resembles the void or volume within an ice cream cone. However, due to the porous nature of the microneedles 20, the CAP 16 is still able to exit the microneedles 20 and penetrate into tissue 100 by passing through the porous material of the microneedles.

The delivery device 14 for the delivery of CAP 16 includes a gas nozzle 30 having an outlet 32 (seen in FIGS. 1A and 1B) from which CAP 16 is ejected. As explained herein, the outlet 32 of the gas nozzle 30 is, during use, located adjacent to the back of the patch 12 (side opposite hollow microneedles 20). The gas nozzle 30 and outlet 32 may be located in a dispensing head 64 such as that illustrated in FIG. 2. In some embodiments, the gas nozzle 30 (or dispensing head 64) may be secured to the patch 12 using a fastener or coupler device that physically connects the gas nozzle 30 to the patch 12. The fastener may also include bands, straps, or the like that can temporarily couple the gas nozzle 30 to the patch 12. In other embodiments, the gas nozzle 30 may just be physically maintained adjacent to the patch 12 to deliver CAP 16 into the hollow microneedles 20. This may be accomplished, for example, by holding the delivery device 14 in place (e.g., manually or using a fixture or the like) next to the patch 12. Regardless of the interface, CAP 16 is ejected from the outlet 32 of the delivery device 14 and passes through the hollow microneedles 20 and into the tissue 100.

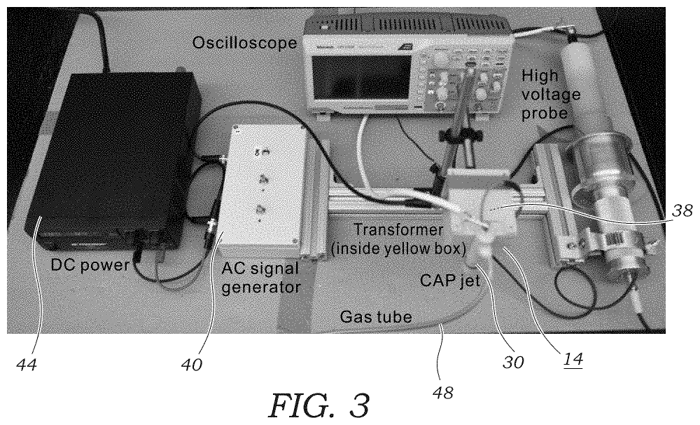

The delivery device 14 has two or more electrodes 34 that are coupled to AC voltage source 36 that is used to create the CAP 16. The two or more electrodes 34 may include a pair (or multiple pairs) of electrodes as illustrated in FIGS. 1A and 1B. The two or more electrodes 34, in the embodiments of FIGS. 1A and 1B, are exposed to the flow path within the nozzle 30 that carries the feed gas (or vapor) 42 (described herein). FIG. 1A illustrates an AC voltage source 36 that is coupled to the two or more electrodes. The AC voltage source 36 may include, as explained herein (e.g., FIG. 3), an AC high voltage transformer 38 powered by an AC signal generator 40 that is coupled the pair of electrodes 34, one of which is a ring electrode (ground electrode) while the second electrode is a needle electrode (powered electrode) as illustrated in FIGS. 1A and 1B. A source of feed gas 42 is coupled to the gas nozzle 30. The feed gas 42 is exposed to the two or more electrodes 34 within a flow path formed within the gas nozzle 30. The source of feed gas 42 may be pressurized so that the feed gas naturally flows through the nozzle 30 in response to pressure. One or more valves and/or gas regulators (not shown) may couple the nozzle 30 to the source of feed gas 42. The source of feed gas 42 may include gas or vapor that includes one or more of helium, air, neon, krypton, argon, oxygen, water (e.g., water vapor), or nitrogen and mixtures thereof

With reference to FIG. 1A, in preferred embodiments, the microneedles 20 and/or the base 18 of the patch 12 are loaded with one or more immune checkpoint inhibitors 43. Exemplary immune checkpoint inhibitors 43 include, for example, ipilimumab, nivolumab, pembrolizumab, avelumab, atezolimumab, durvalumab. These immune checkpoint inhibitors are contained within the matrix of the material that forms the microneedles 20 and are released upon exposure to tissue 100 during use. As explained herein, the microneedles 20 are formed from a biocompatible polymer or hydrogel materials including, for example, a mixture of polyvinylpyrrolidone (PVP) and polyvinyl alcohol (PVA).

FIG. 3 illustrates a photograph showing a prototype delivery device 14 for the delivery of CAP 16. Here, a DC power source 44 is used to power the AC signal generator 40 which is coupled to an AC high voltage transformer 38 enclosed within a housing 46 that contains the nozzle 30 and electrodes 34. The nozzle 30 may be 3D printed or otherwise fabricated using known techniques and integrated with or secured to the housing 46. The gas nozzle 30 includes an inlet coupled to a gas tube or conduit 48 that is coupled to a source of feed gas 42. A needle electrode 34 is located centrally within the gas nozzle 30 while the ring electrode 34 is located in the gas nozzle 30 and surrounds the needle electrode 34. The gas nozzle 30 is positioned to create a jet or stream of CAP 16 that exits from the outlet 32. During use, the gas nozzle 30 is placed immediately adjacent to the backside of the patch 12 (or in some embodiments secured to the patch 12) whereby a jet or stream of CAP 16 exits the gas nozzle 30 and passes through the hollow microneedles 20 and then exits the microneedles 20 after passing through the porous material thereof. There may be a small gap between the exit or outlet of the gas nozzle 30 and the backside of the patch 12. In other embodiments, the gas nozzle 30 may physically contact the backside of the patch 12 directly or through an interface, manifold, or seal. In the prototype of FIG. 3, a high voltage probe and oscilloscope were used to measure discharge voltage. The probe and oscilloscope would not be needed in a commercial embodiment.

In addition, the functions of the DC power source 44 and the AC signal generator 40, in a commercial embodiment, may be combined or integrated into a single or integrated power supply unit 60 such as that illustrated in FIG. 2. FIG. 2 illustrates one embodiment of the delivery device 14 in the form of a hand-held delivery device 14 that includes a handle 62 and a dispensing head 64 that contains the nozzle 30 and outlet 32 for the CAP 16. The power supply 60 may be powered by batteries or using a conventional AC wall socket. In some embodiments, the power supply 60 may be integrated directly into the delivery device 14 (e.g., the handle 62 of the hand-held delivery device 14). As seen in FIG. 2, the source of feed gas 42 is coupled to the hand-held delivery device 14 using tubing or a conduit 66. FIG. 2 illustrates CAP 16 being ejected from the dispensing head 64 and onto a patch 12 that has been adhered to skin tissue 100 of to subject. The hand-held delivery device 14 may include an actuator 68 (e.g., button, trigger, switch) that is triggered/actuated to dispense CAP 16 from the dispensing head 64.

The plurality of hollow microneedles 20 may be formed from a number of biocompatible polymers or hydrogel materials including, for example, a mixture of polyvinylpyrrolidone (PVP) and polyvinyl alcohol (PVA). Additional materials may be used to form the patch including, for example, hyaluronic acid, chitosan, maltose, cellulose, poly(acrylic acid), polylactide acid, poly(lactic-co-glycolic acid), poly(ethylene glycol), or mixtures thereof. The plurality of hollow microneedles 20 may have a number of heights (measured from base 18 to tops 22). For example, the height of the microneedles 20 may be within a range of about 200 μm to about 1.5 mm in one embodiment. In another embodiment, the microneedles 20 may have a height of around 500 μm. The height of microneedles 20 within a single patch 12 may be the same or different. FIG. 1A schematically illustrates the transdermal combinational CAP and ICB therapy assisted by the polymeric hollow-structured microneedle patch 12 loaded with aPDL1 as the immune checkpoint inhibitor 43. Th cell, T helper cell; Tc cell, cytotoxic T cell; TCR, T-cell receptor; MHC, major histocompatibility complex are also illustrated.

To use the patch 12, the patch is applied to the tissue 100 (e.g., skin tissue 100 as seen in FIG. 2) and the plurality of hollow microneedles 20 penetrate the tissue 100. The patch 12, in one embodiment, is preferably located to place the plurality of microneedles 20 directly into or immediately adjacent to the diseased tissue 100. CAP 16 is generated by the flow of feed gas from the source of feed gas 42 past the needle electrode 34 (which is the active electrode in this embodiment) and passes through into the hollow region or void 24 of the hollow microneedles 20 and exits the microneedles 20 into the tissue 100. Small “jets” of CAP 16 may be formed at the tip 22 of each hollow microneedle 20 to aid in penetrating the CAP 16 into the tissue 100. While the patch 12 is applied to the tissue 100 one or more immune checkpoint inhibitors 43 contained in the hollow microneedles 20 are released into the tissue 100. The release of the one or more immune checkpoint inhibitors 43 occurs naturally and after the patch 12 has been adhered to the tissue 100. The immune checkpoint inhibitors 43 may be release as a bolus or over an extended period of time. The release kinetics and characteristics may be controlled by the nature of the materials used for the hollow microneedles 20 (e.g., degree of crosslinking, polymer constituents, etc.). After a period of time, the patch 12 is then removed after application. In other alternative embodiments, the patch 12 may not include any immune checkpoint inhibitors 43 contained in the hollow microneedles 20 and instead the patch 12 is used to deliver only CAP 16 into the tissue 100.

The patch 12 may be applied to the tissue 100 for a number of seconds, minutes, hours, or even longer than a day prior to removal. In some embodiments, a majority of the one or more immune checkpoint inhibitors 43 is released into tissue within twenty-four (24) hours of adhering to the tissue 100. In some embodiments, the patch 12 may remain adhered to the skin tissue 100 after CAP 16 has been introduced into the tissue 100 via the plurality of hollow microneedles 20. For example, the patch 12 may be applied to the skin tissue 100 and CAP treatment takes place for a period of time (e.g., several minutes) and the patch 12 remains in place to release the one or more immune checkpoint inhibitors 43 into the tissue 100. The patch 12 may remain, for example, for several hours or over one or more days. Multiple CAP treatments may be performed on a single patch 12 over an extended period of time.

The delivery device 14 is preferably a separate device from the patch 12 and is interfaced with the patch 12 only at the time CAP 16 is being delivered to tissue 100. In some embodiments, multiple treatments may be needed with a series of patches 12 being applied to the skin tissue 100. These may include patches 12 holding the same or different immune checkpoint inhibitors 43. The different patches 12 may also include different concentrations of immune checkpoint inhibitors 43. Alternatively, the same patch 12 may be used with multiple sessions of CAP 16 being delivered to the tissue 100 using the same patch 12.

During the delivery of CAP 16, the operating parameters of the CAP delivery device 14 may be adjusted to tune the concentration of reactive oxygen and nitrogen species (RONS). The adjustable operating parameters include, for example, the voltage that is applied to the two or more electrodes 34 as well as the frequency (e.g., AC frequency). In some embodiments, the CAP 16 is delivered below the stratum corneum of skin tissue 100. In some embodiments, the patch 12 may be held in place on the skin or other tissue 100 with a bandage, wrap, or an adhesive to keep the patch 12 secured to the skin during the treatment.

### Experimental

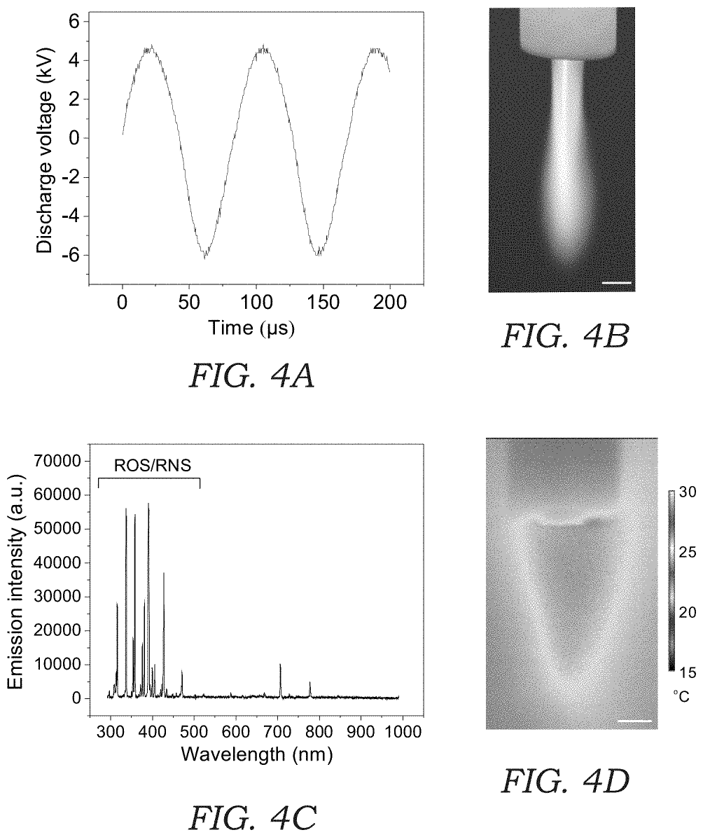

The CAP delivery device 14 (FIG. 3) used for the experiments described herein consists of a two-electrode 34 assembly connected to the high voltage transformer 38. The parameters of the CAP delivery device 14 for stable operation and high species delivery efficiency are as follows: peak-peak voltage ˜11 kV and discharge frequency ˜12 kHz (FIG. 4A). A feeding gas may be used, and for this result industrial purity helium (99.996% purity) was used at a 16.5 L/min gas flow rate. The optical emission spectrum (OES) indicated the generation of both ROS and RNS, while optical and temperature monitoring confirmed the formation of CAP (FIG. 4B-4D).

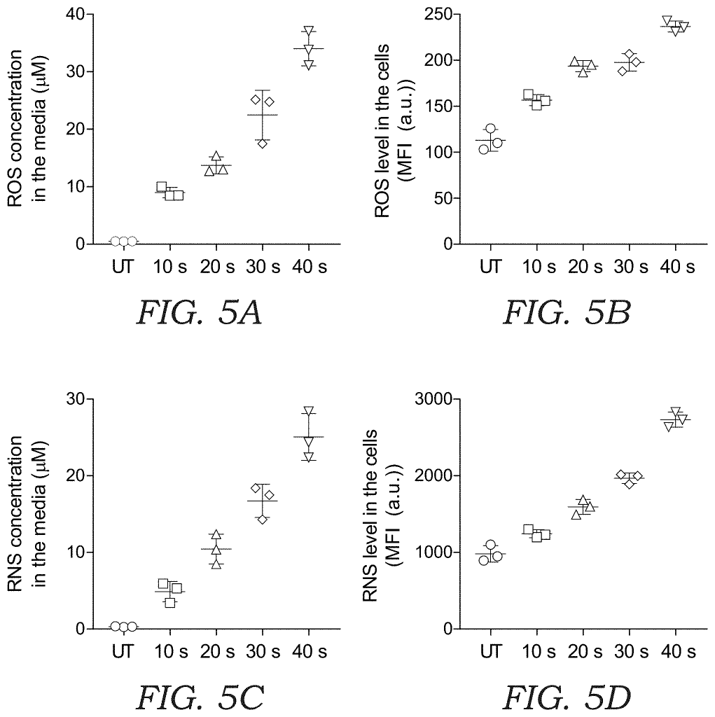

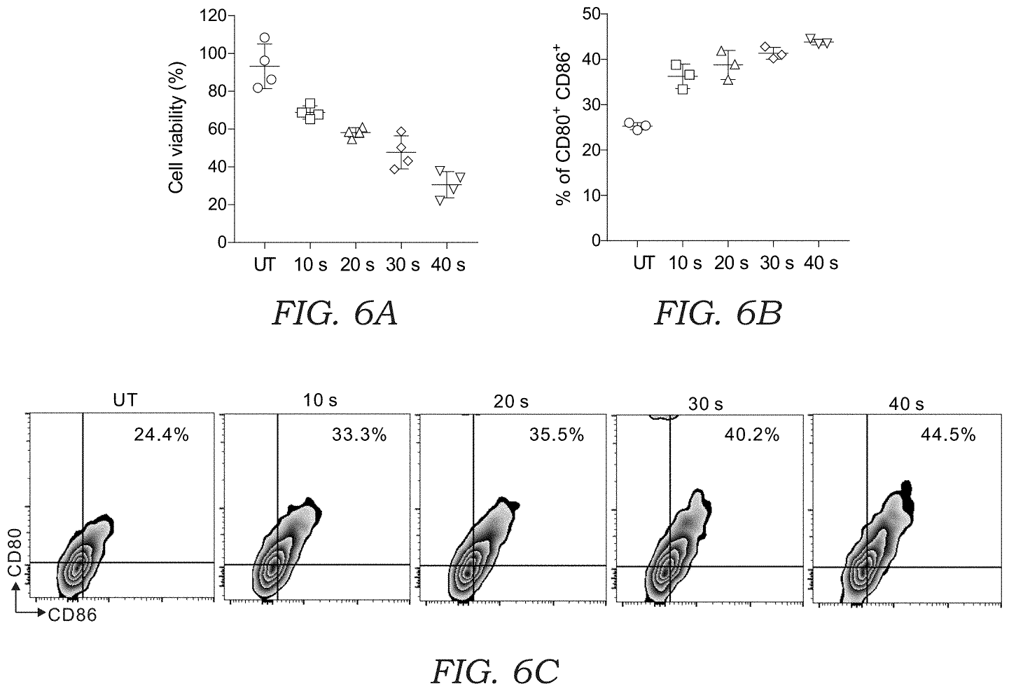

After CAP treatment, ROS and RNS were clearly detected in both cells and culture media, and the extents were elevated with increased treatment time (FIGS. 5A-5D). Cell death induced by CAP 16 delivery was validated in Bl6F10 melanoma cells. CAP 16 clearly caused cancer cell death, which was correlated with the ROS/RNS generation profile that a longer CAP treatment led to a higher cell death rate (FIG. 6A). Tumor-associated antigens released during cell death could be effectively engulfed by immature DCs that process antigens into peptides during their migration to tumor-draining lymph nodes (21, 22). Therefore, the immunological effects of CAP on cancer cells towards bone marrow-derived DCs were studied in a transwell system. It was verified that CAP treatment of Bl6F10 cells greatly promoted DC maturation in vitro (FIGS. 6B and 6C) as indicated by CD80 and CD86 markers.

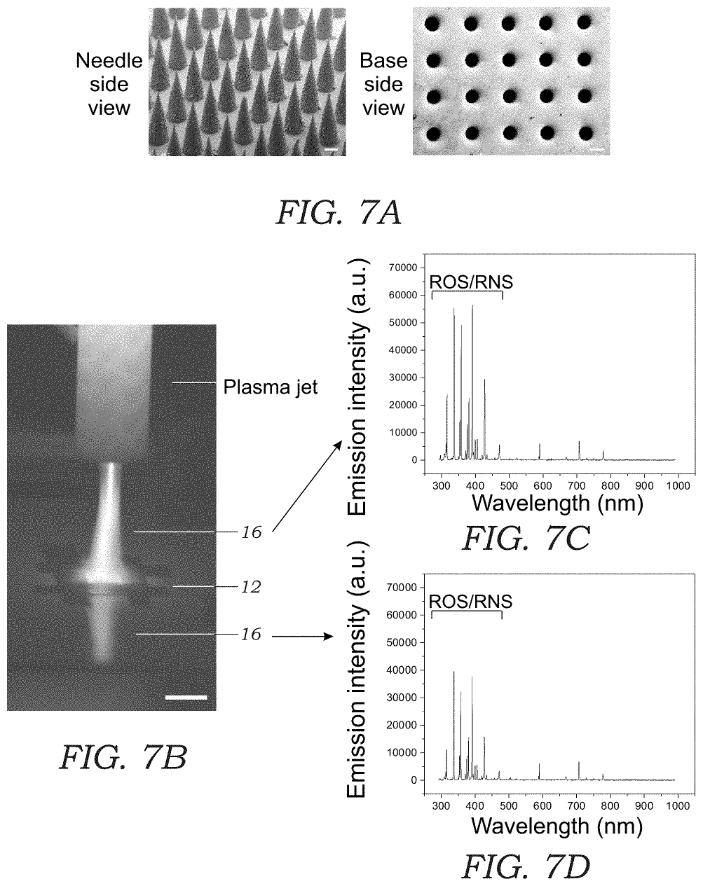

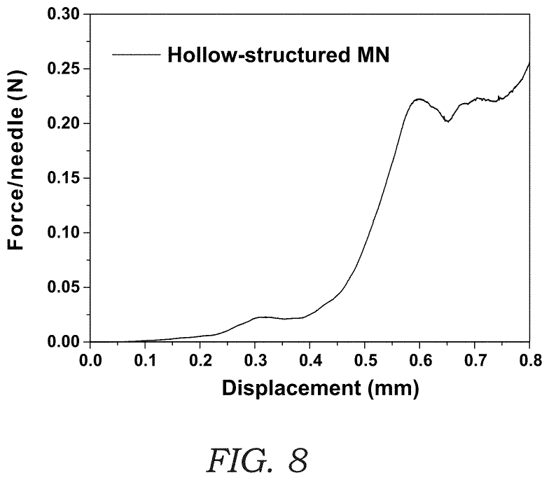

The hollow microneedle patch 12 was made of the mixture of two biocompatible polymers, polyvinylpyrrolidone (PVP) and polyvinyl alcohol (PVA), where PVP supports the strong mechanical strength of MNs 20 and PVA slows down the dissociation of MN 20 patches upon fluids. An array of 15×15 MNs 20, with each MN 20 spaced 600 um center-to-center, was used for all tests. Each MN 20 was conical with a height of 700 μm and a diameter of 300 μm at the base. SEM images show the formation of an evenly-distributed array of equally-sized hollow conical MN structures 20 that comprise the hollow microneedle patch 12 (FIG. 7A). Each MN 20 exhibited a hollow structure closed at the tip 22 and with a 200 μm diameter opening at the base 18, indicating a shell thickness of around 50 μm. The hollow structure and uniformity of shell thickness were confirmed by the three-dimensional confocal laser scanning microscopy images. Measurement of mechanical strength suggested a failure force for the hollow-structured MNs 20 to be 0.23 N/needle, demonstrating sufficient strength for skin insertion (FIG. 8).

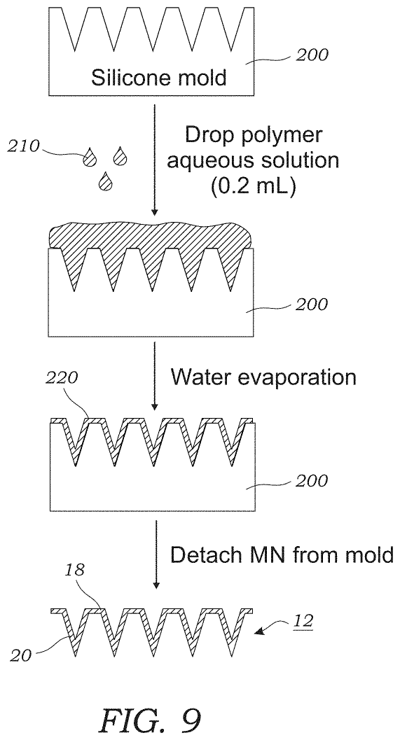

FIG. 9 illustrates an illustrative method for fabricating the MN patch 12. A molding process is used where a silicone mold 200 is provided that has the negative conical surface features (that form the microneedles 20 formed therein). The aqueous polymer solution 210 (with the immune checkpoint inhibitor(s) 43) is deposited on the mold 200 which enters the cervices and spaces that form the microneedles. The water in the solution evaporates leaving a coating 220 that conforms the shape of the mold 200 to create the patch 12 having the microneedles 20 that extend therefrom. The microneedle patch 12 is then removed from the mold 200.

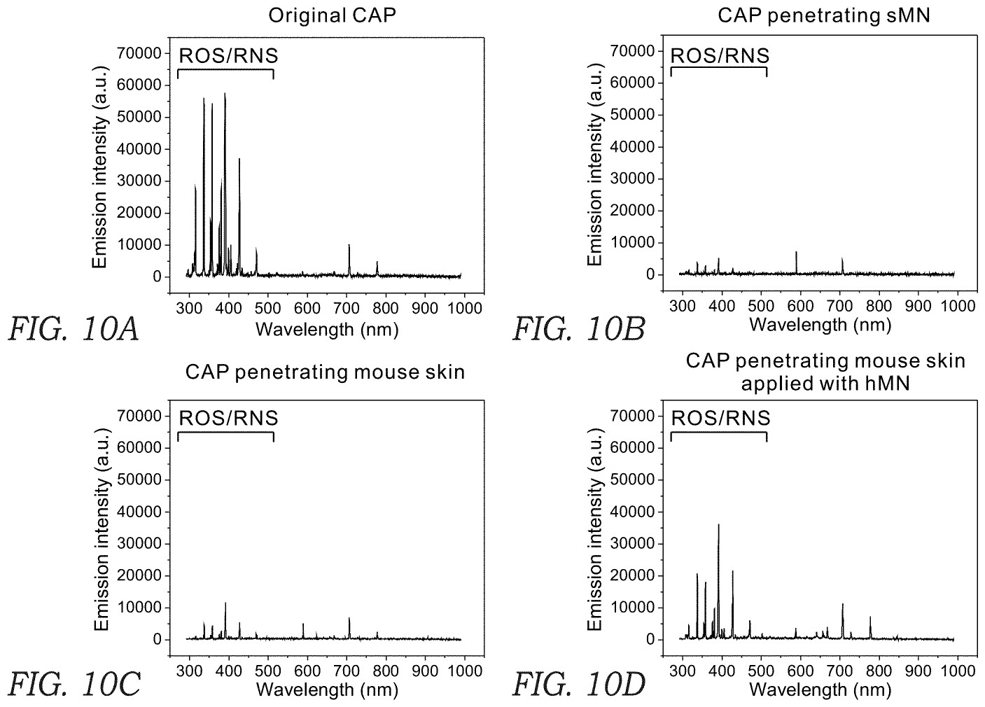

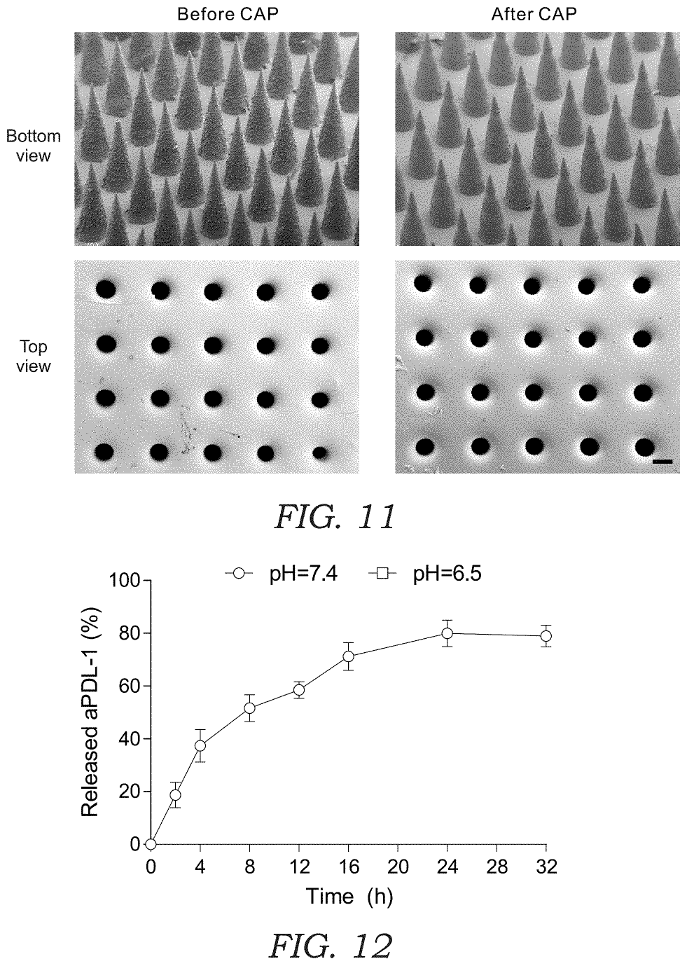

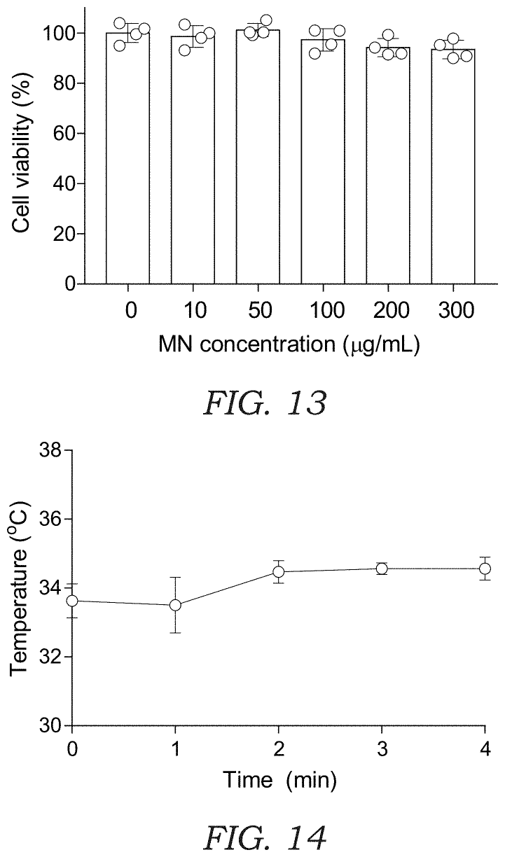

It was then investigated whether the hollow microneedle patch 12 could serve as microchannels to facilitate CAP 16 penetration. A CAP jet was applied to the hollow microneedle patch 12. Strong CAP observation under the hollow microneedle patch 12 (FIG. 7B) indicated successful penetration of CAP 16 through the hollow microneedle patch 12. The OES also confirmed that CAP 16 penetrated through a hollow microneedle patch 12 exhibited a similar composition compared to the original CAP 16, with significant amounts of ROS/RNS retained (FIGS. 7C and 7E). In contrast, minimal CAP signals were detected when the solid microneedle (sMN) patch 12 or the mouse skin was applied (FIG. 10B). On the other hand, the CAP signals penetrated through the mouse skin were greatly enhanced when the hollow microneedle patch 12 was applied (FIG. 10D), demonstrating a beneficial role of hollow microneedle patch 12 for CAP delivery. In the meantime, CAP 16 did not alter the morphology of MN patches 12 as revealed by SEM images (FIG. 11). Hollow microneedle patches 12 can also be embedded with aPDL1 during MN patch 12 fabrication. aPDL1 was released from a hollow microneedle patch 12 in a sustained manner under moderate shaking with 79% being released within 24 h (FIG. 12). hMN patches with blank MN exhibited minimal cytotoxicity to the cells (FIG. 13).

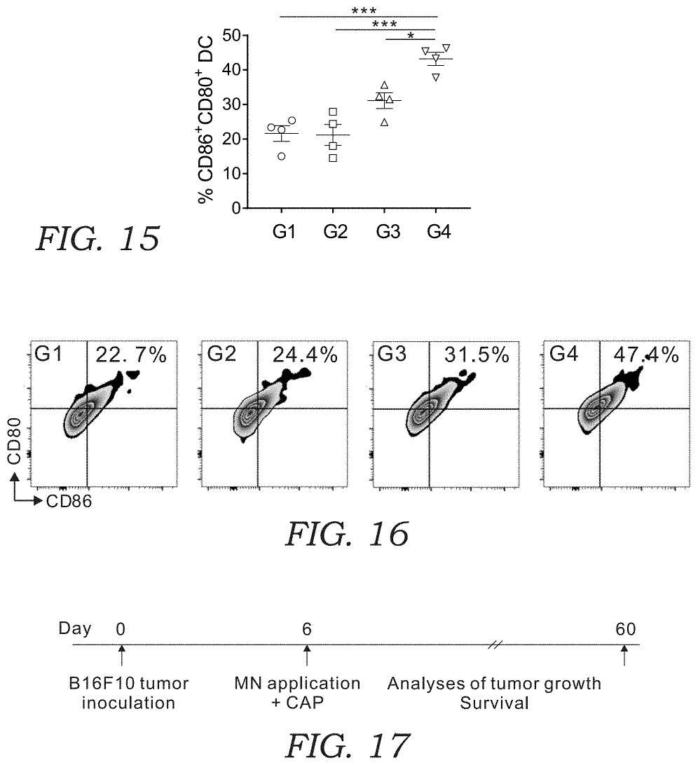

On the basis of the above results, the in vivo performance of this platform was then evaluated. First, it was tested whether hollow microneedle-assisted CAP 16 could induce cell death and trigger DC maturation in Bl6F10 melanoma-bearing mice. Mice received a one-course treatment of CAP, CAP through sMN (sMN/CAP), or CAP through hollow microneedles (hMN) (hMN/CAP) (CAP treatment: 4 min). No temperature changes were detected in the CAP-treated areas, excluding the photothermal cell-killing effect (FIG. 14). The TUNEL assay demonstrated that hMN/CAP treatment induced significant higher cancer cell death than others, indicating that hMN facilitated CAP transportation towards tumors to destroy cells in vivo. Consistently, a clear increased level of DC maturation (CD86⁺CD80⁺DCs) was observed in the hMN/CAP treatment group, whilst direct CAP or sMN/CAP failed to promote the DC maturation (FIGS. 15 and 16).

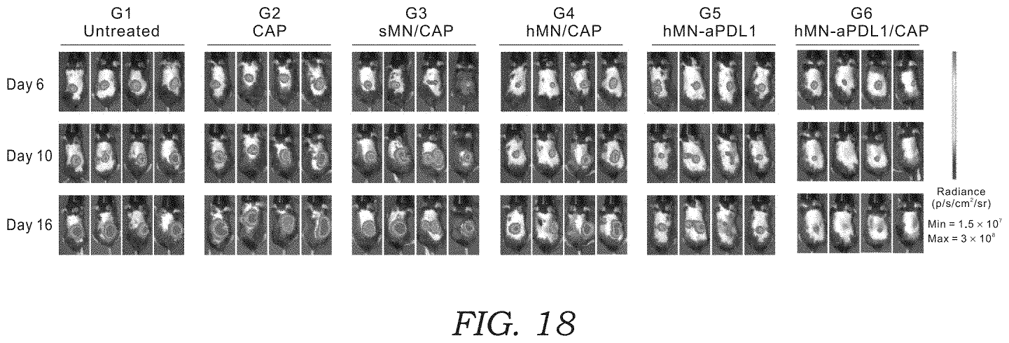

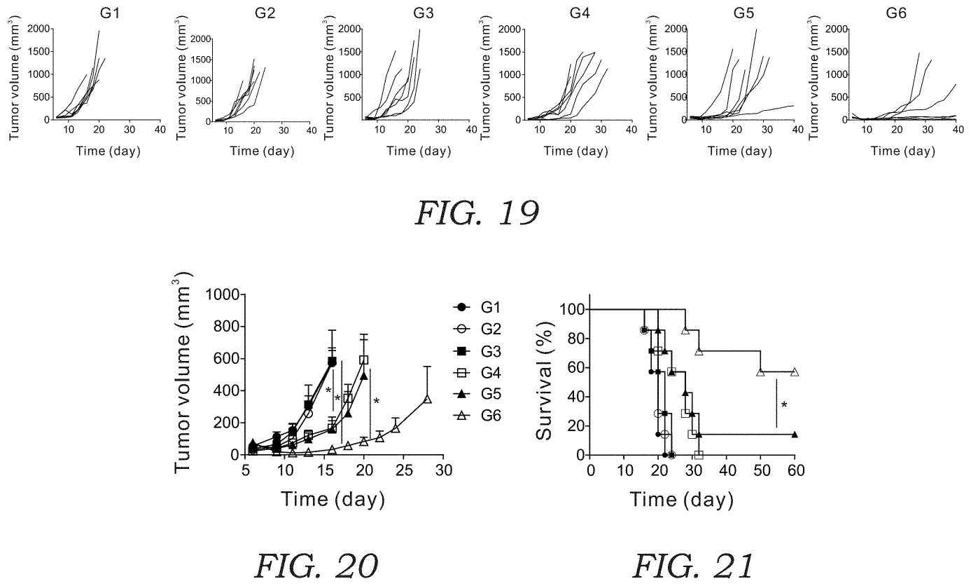

DC maturation can then initiate the T cell-mediated immune response for cancer immunotherapy. Hence, the in vivo anti-cancer effect of the combinational CAP and ICB therapy was tested. In the same mouse tumor model, mice were given a single course of CAP, sMN/CAP, hMN/CAP, hMN-aPDL1, and hMN-aPDL1/CAP (aPDL1: 200 μg; CAP treatment: 4 min; FIG. 17). Tumor growth was monitored via the bioluminescence signals of Bl6F10-fLuc cells (FIG. 18). Neither CAP nor sMN/CAP was superior to the untreated group, again suggesting the limited CAP penetrating capabilities through the skin or sMN, while hMN/CAP showed delayed tumor growth compared to the untreated group (FIGS. 19 and 20). Mice applied with hMN-aPDL1 also exhibited improved anti-cancer efficacy compared with the untreated group. In contrast, mice treated with hMN-aPDL1/CAP exhibited the best control of tumor growth among all treatment groups. Correspondingly, a significantly prolonged survival was observed in mice receiving hMN-aPDL1/CAP treatment (— 57%, FIG. 21).

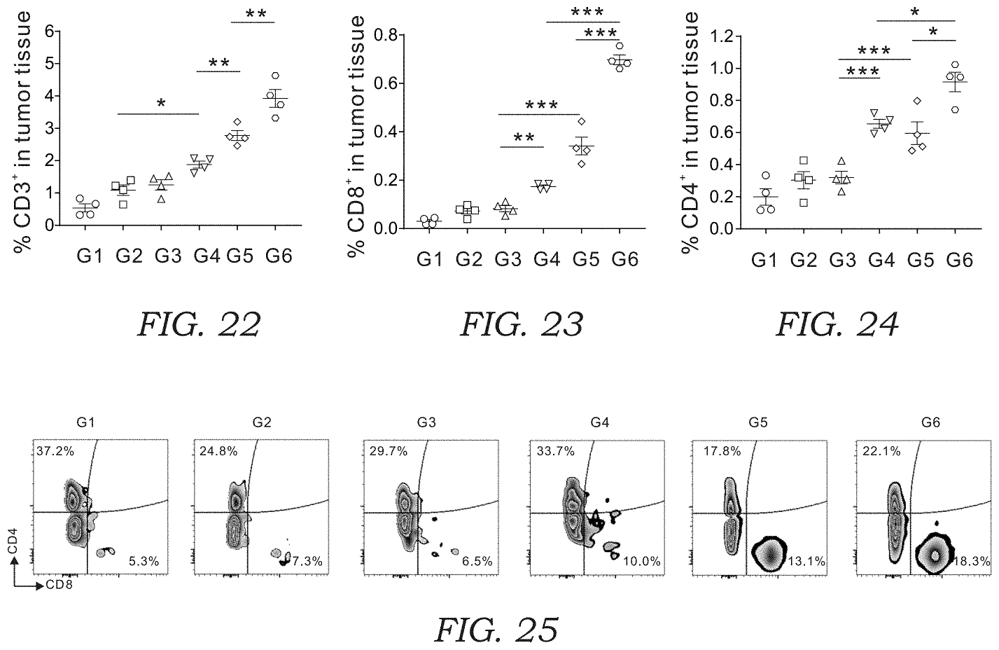

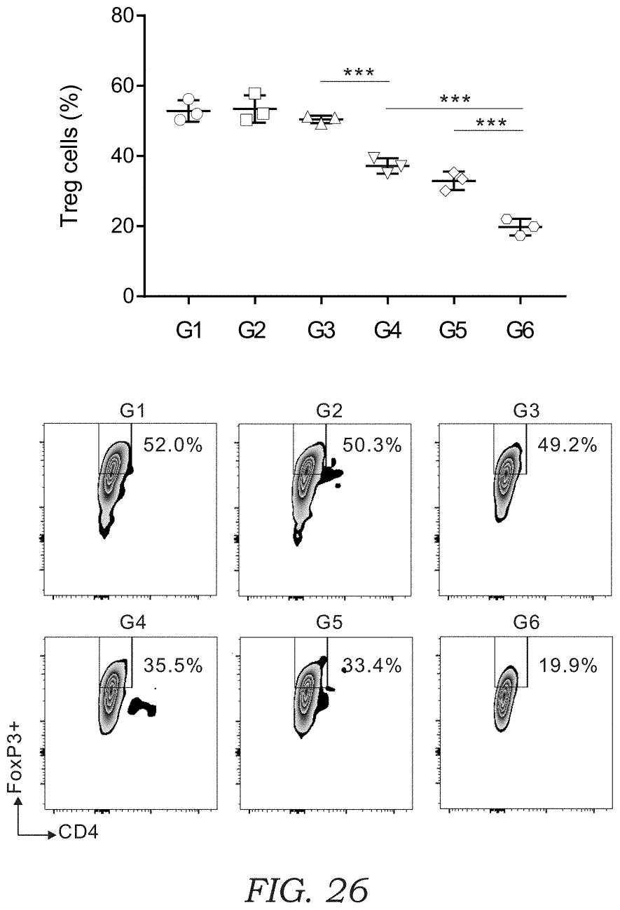

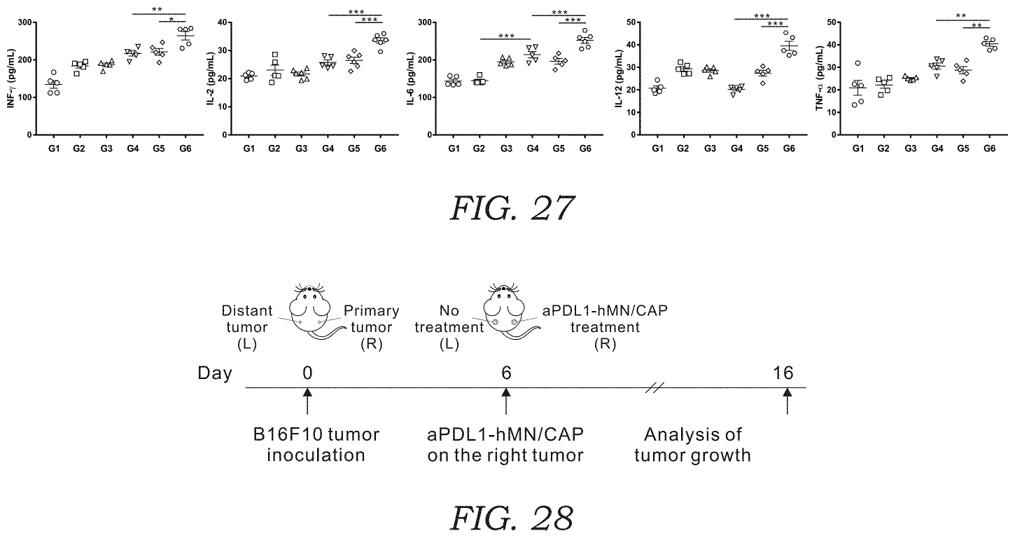

Tumors were harvested three days post-treatment for flow cytometric analyses and immunofluorescence staining. The percentage of tumor-infiltrating lymphocytes (TILs, CD3⁺) was increased in the tumors treated with hMN-aPDL1/CAP (FIG. 22). It also induced marked infiltration of CD8⁺ and CD4⁺ T cells (FIGS. 23, 24 and FIG. 25) compared with other groups, while the number of regulatory T cells (Tregs) was decreased (FIG. 26). The elevated levels of cytokine secretion, including IFN-γ, IL-6, IL-12p70, IL-2, and TNF-α, further substantiated the effective immune response induced by hMN-aPDL1/CAP treatment (FIG. 27).

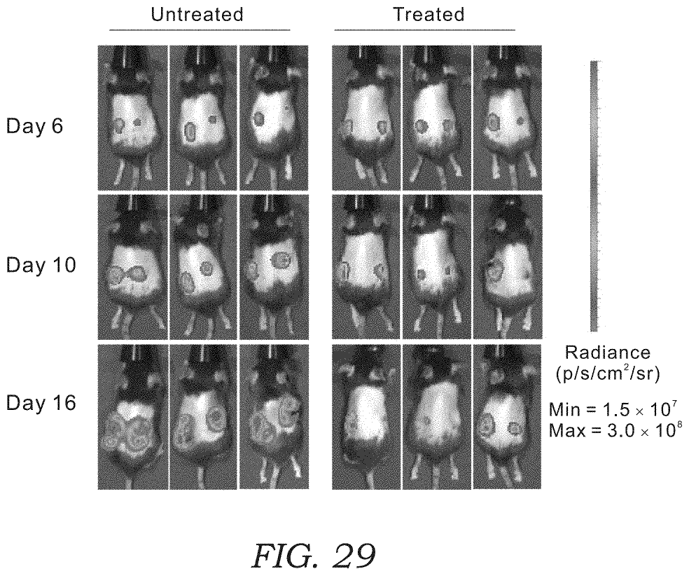

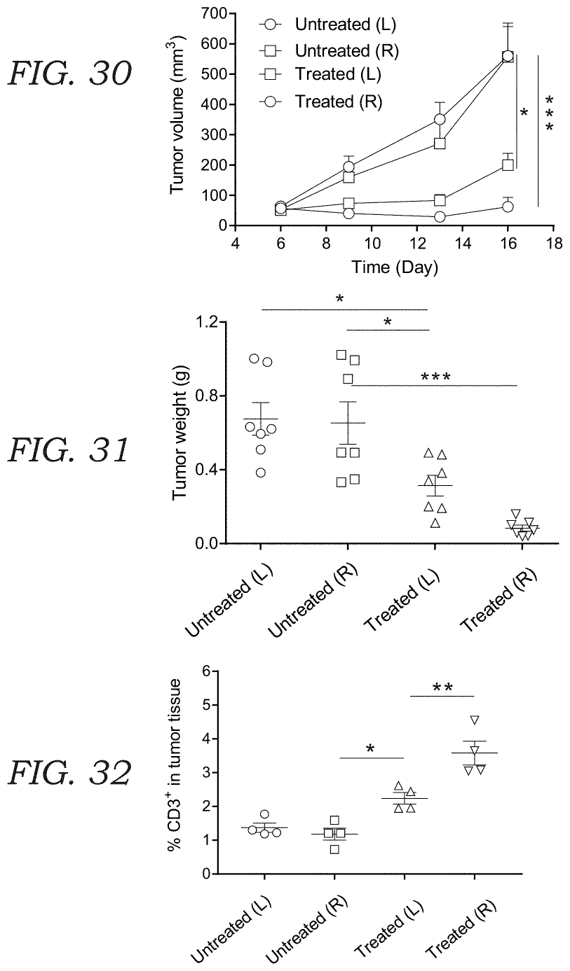

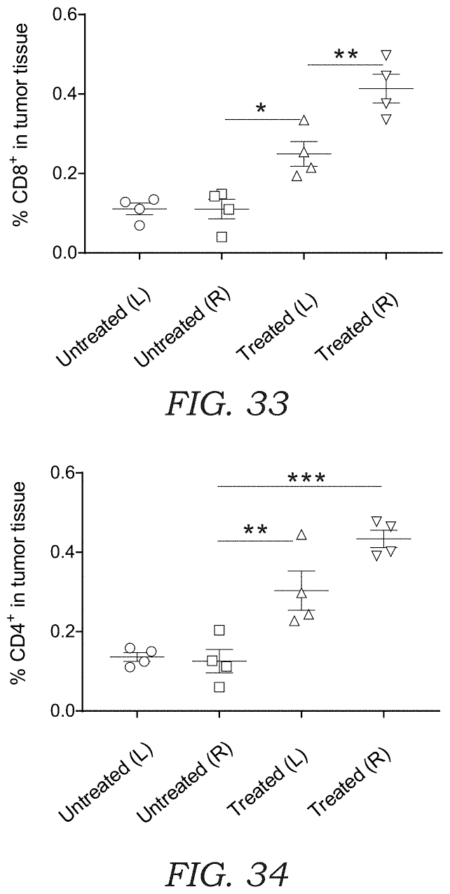

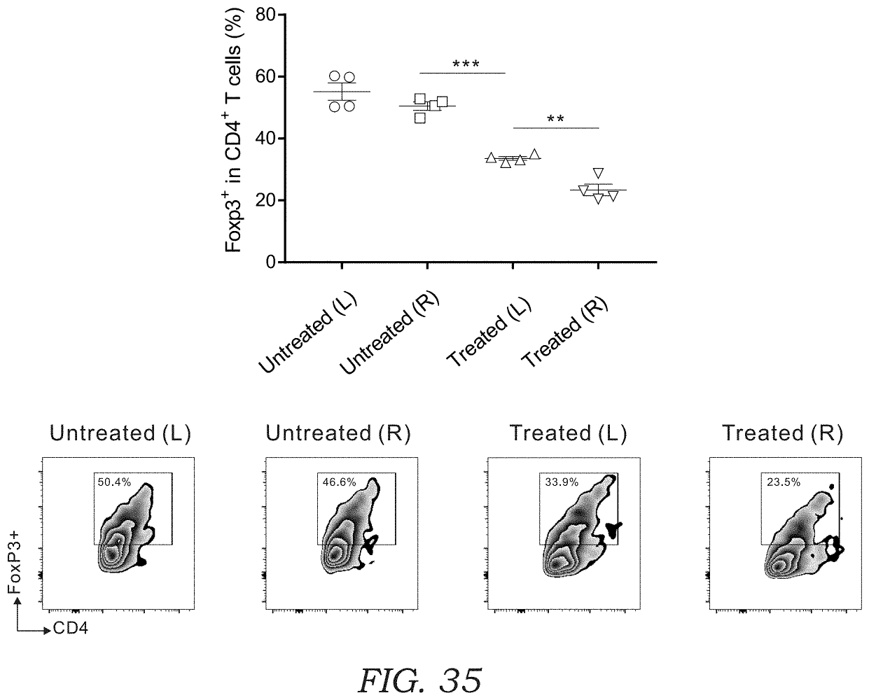

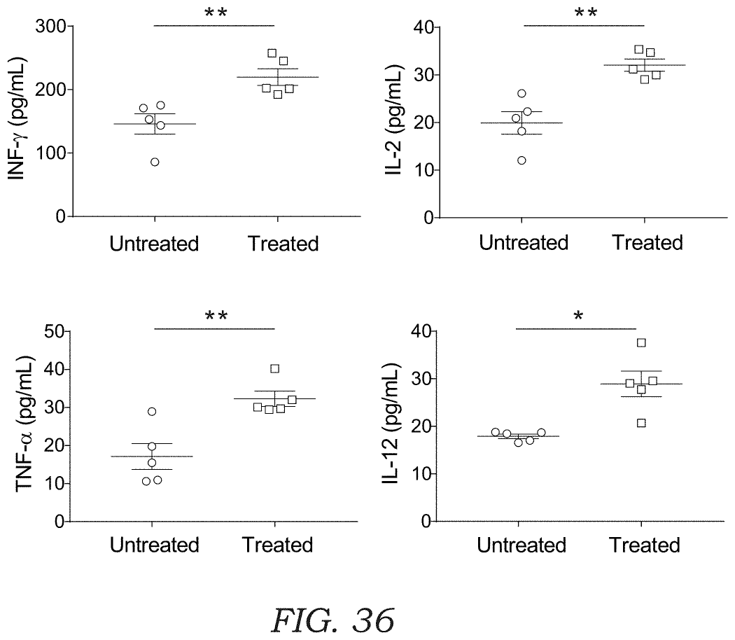

With confirmation that hMN-aPDL1/CAP induced locally anti-cancer immunity, it was investigated whether the local effect induced by hMN-aPDL1/CAP could trigger a systemic immune response against distant tumors. Bl6F10 cancer cells were inoculated on both right and left flanks of each mouse. The tumor on the right flank as the primary tumor was treated with hMN-aPDL1/CAP, while the distant tumor on the opposite site received no treatment to mimic distant tumors (FIG. 28). The bioluminescence signal from the tumors and size of the tumors significantly decreased in the mice treated hMN-aPDL1/CAP compared with untreated controls (FIGS. 29 and 30). More importantly, the left tumors (distant tumors) in the treated mice were also effectively inhibited compared with those in the non-treated mice. Consistently, the weights of primary and distant tumors in the treated mice were also lower than those in the untreated mice (FIG. 31). The increased numbers of TILs (CD3⁺), CD4⁺ and CD8⁺ T cells (FIGS. 32-34), a decreased number of Tregs (FIG. 35), and elevated levels of cytokine secretion (FIG. 36) in both treated tumors and distant tumors confirmed the activation of a systematic immune response.

Leveraging microneedles 20 for transdermal drug delivery, the hollow-structured microneedle patch 12 forms microchannels to deliver CAP 16 through the skin tissue 100 (or other tissue) to enhance the cancer-killing effect. The resulting antigen-presenting by DCs and T cell-mediated immune response augmented by immune checkpoint inhibitors 43 from the hollow microneedle patch 12 further boost anti-cancer immunity locally and systemically. Of note, integrated with the latest microneedle-assisted treatments beyond skin-associated diseases, this method can be extended to treat different cancer types and a variety of diseases.

While embodiments of the present invention have been shown and described, various modifications may be made without departing from the scope of the present invention. The invention, therefore, should not be limited except to the following claims and their equivalents.

1. A patch for cancer or other therapeutic treatment comprising:
a base having a plurality of hollow microneedles extending away from the surface of the base, wherein the plurality of hollow microneedles each have a hollow space or void extending partially through the respective microneedles, wherein the plurality of hollow microneedles further comprise one or more immune checkpoint inhibitors contained therein.

2. The patch of claim 1, further comprising a source of cold atmospheric plasma (CAP) in fluid communication with the respective hollow spaces or voids of the hollow microneedles.

3. The patch of claim 2, wherein the source of cold atmospheric plasma (CAP) comprises a delivery device having a gas nozzle or dispensing head disposed adjacent to the base of the patch and having two or more electrodes coupled to a high voltage source.

4. The patch of claim 1, wherein the plurality of hollow microneedles have a height within the range of about 200 μm to about 1.5 mm.

5. (canceled)

6. The patch of claim 1, wherein the base and the plurality of hollow microneedles comprise a mixture of two or more of polyvinylpyrrolidone (PVP), polyvinyl alcohol (PVA), hyaluronic acid, chitosan, maltose, cellulose, poly(acrylic acid), polylactide acid, poly(lactic-co-glycolic acid), poly(ethylene glycol).

7. The patch of claim 1, wherein the one or more immune checkpoint inhibitors comprises anti-PDL1 antibody (aPDL1).

8. The patch of claim 1, wherein the one or more immune checkpoint inhibitors comprises is selected from the group consisting of ipilimumab, nivolumab, pembrolizumab, avelumab, atezolimumab, durvalumab.

9. The patch of claim 3, wherein the gas nozzle or dispensing head is coupled to a source of feed gas or vapor comprising one or more of helium, air, neon, krypton, argon, oxygen, water, or nitrogen and combinations thereof.

10. A method of using the patch of claim 1 comprising:
adhering the patch on tissue wherein the plurality of hollow microneedles penetrate the tissue;
introducing cold atmospheric plasma (CAP) into the hollow spaces or voids of the hollow microneedles and into the tissue with a delivery device; and
releasing the one or more immune checkpoint inhibitors contained in the hollow microneedles into the tissue.

11. The method of claim 10, wherein the one or more immune checkpoint inhibitors comprises anti-PDL1 antibody (aPDL1).

12. The method of claim 10, wherein the one or more immune checkpoint inhibitors is selected from the group consisting of ipilimumab, nivolumab, pembrolizumab, avelumab, atezolimumab, durvalumab.

13. The method of claim 10, wherein the tissue comprises skin tissue.

14. The method of claim 10, wherein the patch is used to treat cancer.

15. The method of claim 10, wherein the patch is used to treat skin cancer.

16. The method of claim 10, wherein a majority of the one or more immune checkpoint inhibitors is/are released into tissue within 24 hours of adhering to the tissue.

17. The method of claim 10, wherein the delivery device is secured to the patch.

18. The method of claim 10, wherein the delivery device is held adjacent to the patch.

19. A patch for cancer or other therapeutic treatment comprising:
a base having a plurality of hollow microneedles extending away from the surface of the base, wherein the plurality of hollow microneedles each have a hollow space or void extending partially through the respective microneedles.

20. A system for treatment of cancer or other neoplasms in mammals comprising:
a patch comprising a plurality of hollow microneedles, wherein the plurality of hollow microneedles each have a hollow space or void extending partially through the respective microneedles, wherein the plurality of hollow microneedles further comprise one or more immune checkpoint inhibitors contained therein; and
a cold atmospheric plasma (CAP) delivery device comprising a gas nozzle or dispensing head having two or more electrodes operatively coupled to a high voltage source.

21. The system of claim 20, wherein the delivery device comprises a hand-held delivery device.

22. The system of claim 20, further comprising a source of feed gas or vapor fluidically coupled to the gas nozzle or dispensing head.

23. The system of claim 20, wherein the one or more immune checkpoint inhibitors is selected from the group consisting of ipilimumab, nivolumab, pembrolizumab, avelumab, atezolimumab, durvalumab, and anti-PDL1 antibody (aPDL1).

24. The system of claim 20, wherein the plurality of hollow microneedles comprise a mixture of two or more of polyvinylpyrrolidone (PVP), polyvinyl alcohol (PVA), hyaluronic acid, chitosan, maltose, cellulose, poly(acrylic acid), polylactide acid, poly(lactic-co-glycolic acid), poly(ethylene glycol).
